(19) United States
(12) Patent Application Publication
(10) Pub. No.: US 2017/0156605 A1
(43) Pub. Date: Jun. 8, 2017

(54) IMAGING APPARATUS AND IMAGING METHOD

(71) Applicant: Sony Corporation, Tokyo (JP)

(72) Inventors: Isamu Nakao, Tokyo (JP); Takuya Kishimoto, Tokyo (JP)

(73) Assignee: Sony Corporation, Tokyo (JP)

(21) Appl. No.: 15/325,558

(22) PCT Filed: Jun. 22, 2015
(86) PCT No.: PCT/JP2015/003103

§ 371 Date: Jan. 11, 2017

(30) Foreign Application Priority Data
Jul. 18, 2014 (JP) 2014-147733

(51) Int. Cl. A61B 5/026; G06T 7/00; G06T 5/50; A61B 5/00

(52) U.S. Cl. CPC A61B 5/0261; A61B 5/7278; G06T 7/0012; G06T 5/50; A61B 5/0066; A61B 2576/02; G06T 2207/20216; G06T 2207/20221; G06T 2207/10021; G06T 2207/30104

(57) ABSTRACT

An imaging apparatus, an imaging method, methods, and image processing apparatuses. There is provided an imaging apparatus including: a light separation unit configured to separate coherent light into object light and reference light, wherein the object light irradiates an object; an optical element configured to cause interference between: scattered light emitted from a region irradiated by the object light and the reference light; an imaging unit configured to image a plurality of interference fringes having different phase differences between the object light and the reference light; and an image formation unit configured to form, from a plurality of images of the plurality of interference fringes, a three dimensional image including a speckle component comprising a random interference or diffraction pattern.

### CROSS REFERENCE TO RELATED APPLICATIONS

This application claims the benefit of Japanese Priority Patent Application JP 2014-147733 filed Jul. 18, 2014, the entire contents of which are incorporated herein by reference.

### TECHNICAL FIELD

The present disclosure relates to a flow channel imaging apparatus and a flow channel imaging method. To be more specific, it relates to a flow channel imaging apparatus and a flow channel imaging method that image a flow channel such as a blood vessel in a three-dimensional manner.

### BACKGROUND ART

In general, a method of acquiring an X-ray image by injecting a contrast medium into a blood vessel is used to confirm the state and position of the blood vessel in a body. Moreover, in recent years, an angiography method that can acquire a three-dimensional image is developed like computed tomography (CT) angiography and magnetic resonance angiography (MRA).

Moreover, there is also suggested a method of imaging a flow channel such as a blood vessel by the use of an optical method in the related art (see PTL 1). The imaging system described in PTL 1 images an interference light image by light with which light from a light emitting unit reflects to an object and interferes at the first timing, and images a luminescence image of light emitted from the object at the second timing. In addition, there is also suggested a method of improving the location accuracy of blood vessels by image processing (see PTL 2).

### CITATION LIST

### Patent Literature

PTL 1: JP 2009-136396A

PTL 2: JP 2013-583A

### SUMMARY

### Technical Problem

However, the above-described flow channel imaging method in the related art has problems that an apparatus is large and it takes time to image a three-dimensional image.

Therefore, mainly, the present disclosure provides a flow channel imaging apparatus and flow channel imaging method that can image a three-dimensional image of a flow channel in a simple method.

### Solution to Problem

An imaging apparatus according to some embodiments may include: a light separation unit configured to separate coherent light into object light and reference light, wherein the object light irradiates an object;

an optical element configured to cause interference between: scattered light emitted from a region irradiated by the object light and the reference light; an imaging unit configured to image a plurality of interference fringes having different phase differences between the object light and the reference light; and an image formation unit configured to form, from a plurality of images of the plurality of interference fringes, a three dimensional image including a speckle component comprising a random interference or diffraction pattern.

Alternatively or additionally, an imaging method according to some embodiments may include: a light separation process of separating coherent light emitted from an optical source into object light and reference light, wherein the object light irradiates an object; an image acquisition process of: causing interference between: scattered light emitted from a region irradiated with the object light and the reference light, and imaging a plurality of interference fringes having different phase differences between the object light and the reference light; and an image formation process of forming, from a plurality of images of the plurality of interference fringes, a three-dimensional image including a speckle component comprising a random interference or diffraction pattern.

Alternatively or additionally, a method according to some embodiments may include: receiving a plurality of three-dimensional images, wherein each three dimensional image of the plurality of three-dimensional images includes a speckle component comprising a random interference or diffraction pattern; determining a spatial distribution of speckle intensity in the plurality of three-dimensional images; and identifying, based on the spatial distribution of speckle intensity, a part of the plurality of three-dimensional images as an object irradiated by coherent light.

Alternatively or additionally, a method according to some embodiments may include: receiving a plurality of images having different phase differences between an object light of a coherent light and a reference light of the coherent light; determining a spatial distribution data of speckle intensity based on the plurality of images at least by performing addition processing or averaging processing on the plurality of images; and identifying, based on the spatial distribution data of speckle intensity, an object irradiated by the coherent light.

Alternatively or additionally, an image processing apparatus according to some embodiments may include: an image processing unit configured to: receive a plurality of three-dimensional images, wherein each three dimensional image of the plurality of three-dimensional images includes a speckle component comprising a random interference or diffraction pattern; determine a spatial distribution of speckle intensity in the plurality of three-dimensional images; and identify, based on the spatial distribution of speckle intensity, a part of the plurality of three-dimensional images as an object irradiated by coherent light.

Alternatively or additionally, an image processing apparatus according to some embodiments may include: an image processing unit configured to: receive a plurality of images having different phase differences between an object light of a coherent light and a reference light of the coherent light; determine a spatial distribution data of speckle intensity based on the plurality of images at least by performing addition processing or averaging processing on the plurality of images; and identify, based on the spatial distribution data of speckle intensity, an object irradiated by the coherent light.

### Advantageous Effects of Invention

According to an embodiment of the present disclosure, it is possible to image a three-dimensional image a simple method since it is possible to acquire three-dimensional position information having a spatial structure by the speckle imaging method. Here, an effect described here is not limited at any time but may be any effect described in the present disclosure.

### BRIEF DESCRIPTION OF DRAWINGS

FIG. 1 is a diagram schematically illustrating the configuration of a flow channel imaging apparatus of the first embodiment of the present disclosure.

FIG. 2 is a diagram illustrating a formation method of a three-dimensional image.

FIG. 3 is a diagram schematically illustrating the configuration of a flow channel imaging apparatus of the second embodiment of the present disclosure.

FIG. 4 is a schematic diagram illustrating a configuration example of a polarization imaging element illustrated in FIG. 3.

FIG. 5 is a schematic diagram illustrating the configuration of a flow channel imaging apparatus of the first example of the present disclosure.

FIG. 6 is a schematic diagram illustrating the configuration of a flow channel imaging apparatus of the second example of the present disclosure.

### DESCRIPTION OF EMBODIMENTS

In the following, modes to implement the present disclosure are described in detail with reference to the accompanying drawings. Here, the present disclosure is not limited to each following embodiment. Moreover, an explanation is given in the following order.

### 1. First Embodiment

### (Example of Flow Channel Imaging Apparatus Including Phase Adjustment Unit)

### 2. Second Embodiment

(Example of Flow Channel Imaging Apparatus Including Solid-state Imaging Element in which One Pixel is Divided into Multiple Areas)

(1. First Embodiment)

First, a flow channel imaging apparatus according to the first embodiment of the present disclosure is described.

The present inventor obtained the following findings as a result of earnestly carrying out an experimental examination to solve the above-described problem. A speckle is a random interference/diffraction pattern by scattering in an optical path, and so on. Moreover, the size of the speckle is shown by an index called a speckle contrast that is the value dividing the standard deviation of intensity distribution by average brightness. When an object illuminated by the use of coherent light is observed by the use of an image formation optical system, a speckle by scattering of the object on an image surface is observed. Further, when the object moves or the shape thereof changes, a random speckle pattern corresponding thereto is observed.

When a light-scattering fluid like blood is observed, a speckle pattern momently changes by a change in a fine shape by flow. At this time, when an imaging element is set up on an imaging surface and a fluid is imaged in exposure time that is sufficiently longer than the change in the speckle pattern, the speckle contrast in a part in which blood flows, that is, the speckle contrast in a blood vessel part decreases by time averaging. Angiography can be performed by using such a change in the speckle contrast. Moreover, when the brightness of one point in the speckle is focused on, the intensity momently changes by flow. When the autocorrelation of this time change is calculated, a short correlation time is acquired in a case where the flow is fast, and a long correlation time is acquired in a case where the flow is slow.

The angiography can also be performed by using such a principle. As a simple method of imaging a flow channel such as blood vessel, the present inventor suggests a fluid analysis apparatus including: a light irradiation unit configured to irradiate a fluid with coherent light; an image formation optical system configured to form the light with which the fluid is irradiated; and a data acquisition unit configured to acquire speckle data of the fluid. In this fluid analysis apparatus, since the numerical aperture of the image formation optical system is adjusted on the basis of the speckle data of the fluid, it is possible to detect the fluid accurately.

Here, speckle contrast data can be used as the speckle data, and, in this case, the image formation optical system adjusts the numerical aperture such that speckle contrast becomes maximum. Moreover, speckle contrast data of others than the fluid may also be acquired by the data acquisition unit, and the numerical aperture may be adjusted such that the difference between the speckle contrast of the fluid and the speckle contrast of others than the fluid becomes maximum in the image formation optical system.

By further conducting an examination for the above-described speckle imaging method and acquiring a three-dimensional image including a speckle by the use of a digital holography method, the present inventor found that it is possible to acquire information in the depth direction of the flow channel even by the speckle imaging, and achieved the present disclosure.

(Whole Configuration)

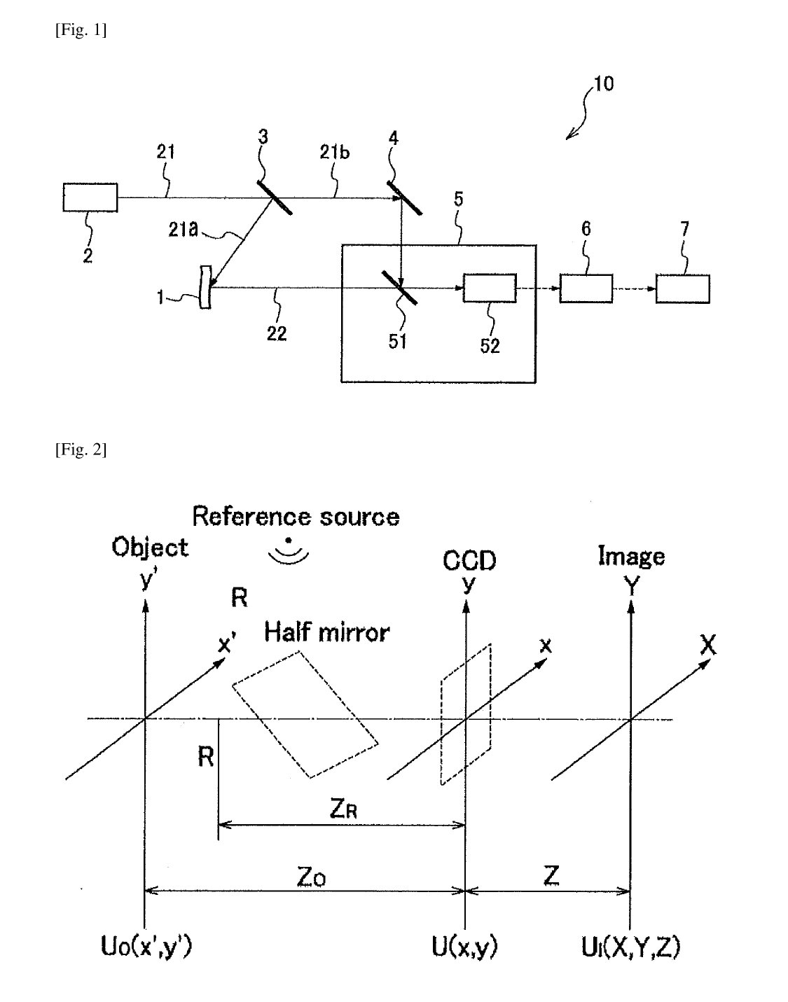

FIG. 1 is a diagram schematically illustrating the configuration of a flow channel imaging apparatus of the present embodiment. As illustrated in FIG. 1, a flow channel imaging apparatus 10 of the present embodiment images a flow channel 1 in three-dimensional manner, and, for example, includes an optical source 2, a light separation unit 3, a phase adjustment unit 4, an imaging unit 5, an image formation unit 6 and an image processing unit 7, and so on.

(Flow Channel 1)

The flow channel 1 is, for example, a blood vessel, and, in that case, a fluid is blood. Additionally, a lymphatic vessel is enumerated as the flow channel 1 imaged by the flow channel imaging apparatus of the present embodiment, and, in that case, the fluid is a lymph fluid. Moreover, application to a visualization technique of various light scattering fluids, and so on, is enumerated as industrial application.

(Optical Source 2)

The optical source 2 only has to be able to emit coherent light 21 and the kind thereof is not especially limited, and, for example, it is possible to use a semiconductor laser, a solid-state laser and a gas laser.

(Light Separation Unit 3)

The light separation unit 3 separates the coherent light 21 into object light 21a with which a fluid 11 that flows through the flow channel 1 is irradiated and reference light 21b, and it is possible to use a beam splitter and a half mirror, and so on. Moreover, it is possible to dispose a dimmer filter, a wavelength plate and a polarizing beam splitter, and so on, in the light separation unit 3 according to the necessity, and the intensity of the object light 21a and the reference light 21b may be changed by these and adjusted such that hologram signals become maximum.

(Phase Adjustment Unit 4)

The phase adjustment unit 4 shifts the phase of reference light 21b. A method of shifting the phase of the reference light 21b is not especially limited, and, for example, it is possible to apply a method using an electro-optical element and a method of changing optical length by the use of a piezoelectric element, and so on. Moreover, it is preferable that the adjustment range of the phase of the reference light 21b by the phase adjustment unit 4 is assumed to be 0 to 2 pi. By this means, since it is possible to acquire hologram images whose phases are shifted by pi/2, it is possible to generate a three-dimensional image by Expression 4 and Expression 5 described later.

(Imaging Unit 5)

The imaging unit 5 causes scattered light 22 emitted from an area irradiated with the object light 21a and the reference light 21b to interfere with each other, images the interference fringe thereof and includes a beam combiner 51 that synthesizes the reference light 21b and the scattered light 22, and an imaging element 52, and so on. Here, a charge coupled device (CCD) and a complementary metal oxide semiconductor (CMOS) sensor, and so on, can be used as the imaging element 52.

(Image Formation Unit 6)

The image formation unit 6 forms a three-dimensional image including a speckle component, from a plurality of images with different phase differences between the object light 21a and the reference light 21b, which are acquired in the imaging unit 5. This image formation unit 6 may be installed in the imaging apparatus 10, or it may be installed in a computer connected with the imaging apparatus 10, and so on.

For example, the image formation unit 6 can form a three-dimensional image with four images in which phase differences between the object light 21a and the reference light 21b are different from each other by pi/2. By this means, the three-dimensional image with a superimposed speckle is acquired. Moreover, it is preferable that the image formation unit 6 forms a three-dimensional image with a plurality of images continuously imaged in a shorter time than a speckle correlation time. By this means, a single three-dimensional image without speckle time average by flow is acquired.

(Image Processing Unit 7)

The image processing unit 7 performs addition processing or averaging processing on a plurality of three-dimensional images formed by the image formation unit 6, and calculates the spatial distribution of speckle contrast. Further, images before and after processing are compared, a part in which the speckle contrast is decreased by the processing is extracted, and the part is assumed to be a flow channel. Thus, by performing the addition processing or the averaging processing, it is possible to specify a part with flow from the averaging of the speckle pattern by flow, that is, from a decrease in the speckle contrast. This image processing unit is also installed in the imaging apparatus 10 or a computer connected with the imaging apparatus 10.

(Operation)

Next, the operation of the above-described flow channel imaging apparatus 10 is described. In the flow channel imaging method of the present embodiment, a plurality of three-dimensional images including a speckle are imaged in shorter exposure time than the correlation time of a speckle formed by a fluid that flows through a flow channel and at longer intervals than the correlation time of the speckle, by the use of a digital holography method. Further, these multiple three-dimensional images are added or averaged to find the spatial distribution of speckle contrast, a part in which the speckle contrast is decreased as compared with the images before the addition or averaging is extracted, and it is assumed as a flow channel.

Specifically, in a case where a three-dimensional image of the flow channel 1 is imaged by the use of the flow channel imaging apparatus 10 of the present embodiment, first, the coherent light 21 emitted from the optical source is separated into the object light 21a and the reference light 21b (light separation process). Afterward, the scattered light 22 emitted from the region irradiated with the object light 21a and the reference light 21b are caused to interfere with each other, and the interference fringe thereof is imaged (image acquisition process). Further, a three-dimensional image including a speckle component is formed with a plurality of images with different phase differences between the object light 21a and the reference light 21b (image formation process), and a plurality of three-dimensional images are subjected to addition processing or averaging processing (image processing process).

For example, the three-dimensional image can be formed in the following method. FIG. 2 is a diagram illustrating a formation method of the three-dimensional image. As illustrated in FIG. 2, the complex amplitudes of the object light 21a and the reference light 21b are expressed by Expression 1 and Expression 2 listed below, respectively.

### [Math.1]

U_{O}=A_{O} exp[iφ_{O}(x,y)] (1)

### [Math.2]

U_{R}=A_{R} exp[iφ_{R}(x,y)] (2)

Moreover, when the phase shift amount is assumed to be delta, the intensity distribution of light received by the imaging element 52 is expressed by Expression 3 listed below.

$$\begin{matrix}[ Math . 3 ] & \\ \begin{matrix}\vert ( x , y , δ ) = {\vert {U}_{R} ( x , y ) exp ( iδ ) + {U}_{0} ( x , y ) \vert}^{2} \\ = {\vert {U}_{R} ( x , y ) \vert}^{2} + {\vert {U}_{O} ( x , y ) \vert}^{2} + \\ {U}_{O} {U}_{R} * exp ( - iδ ) + {U}_{O}^{*} {U}_{R} exp ( iδ )\end{matrix} & ( 3 )\end{matrix}$$

Further, when four kinds of phases of 0, pi/2, pi and 3pi/2 are used, the complex amplitude in the light receiving surface (hologram surface) of an imaging element is expressed by Expression 4 listed below.

$$\begin{matrix}[ Math . 4 ] & \\ U ( x , y ) = \frac{1}{4 {U}_{R}^{*}} \{ I ( x , y , 0 ) - I ( x , y , π ) + i [ ( x , y , \frac{π}{2} ) - I ( x , y , \frac{3 π}{2} ) ] \} & ( 4 )\end{matrix}$$

In the case of a hologram by diffraction in the Fresnel region, the complex amplitude in the position of coordinate z in the optical axis direction is calculated from Expression 5 listed below by performing Fresnel transformation of above-described Expression 4.

$$\begin{matrix}[ Math . 5 ] & \\ UI ( X , Y , Z ) = \int \int U ( x , y ) exp [ i k \frac{{( X - x )}^{2} + {( Y - y )}^{2}}{2 Z} ] d x d y & ( 5 )\end{matrix}$$

A three-dimensional image of the flow channel 1 can be acquired by calculating the intensity of above-described Expression 5. At this time, when the flow channel 1 is illuminated with light with high coherence such as laser light, a speckle is caused on the object surface and the light receiving surface (hologram surface) of an imaging element, and so on, by scattering of the object. Since information is imported with this speckle as a hologram, the speckle is resynthesized as an image even in a case where the image is generated by above-described Expression 5.

Meanwhile, in a case where there is the flow of a light scattering fluid in an object, a speckle pattern to be resynthesized momently changes in the flow channel 1. Therefore, by overlapping and averaging a plurality of resynthesized images, the speckle contrast is decreased in the flow channel 1. Moreover, since the brightness in pixel units in the flow channel 1 changes depending on time, the correlation time becomes shorter than a part that is not a flow channel.

In a case where these are measured, it is preferable that exposure time at imaging by the imaging element 52 is set to be sufficiently shorter time than a speckle correlation time. By this means, it is possible to suppress complex amplitude information from being lost by averaging by flow at imaging.

Since the flow channel imaging apparatus of the present embodiment determines the position of a flow channel by overlapping a plurality of images imaged in a digital holography method and performing addition processing or averaging processing, it is possible to image a three-dimensional image of the flow channel in a simple method. Further, in the flow channel imaging method of the present embodiment, it is also possible to acquire three-dimensional position information on a blood vessel existing in the vicinity of the surface of a living organ having a spatial structure which has been difficult in a speckle blood stream imaging method in the related art. As a result, in the case of performing an operation while watching a video image like a microscope operation and an endoscopic procedure, and so on, blood vessel position information that can be provided for the doctor becomes accurate especially in the depth direction.

(2. Second Embodiment)

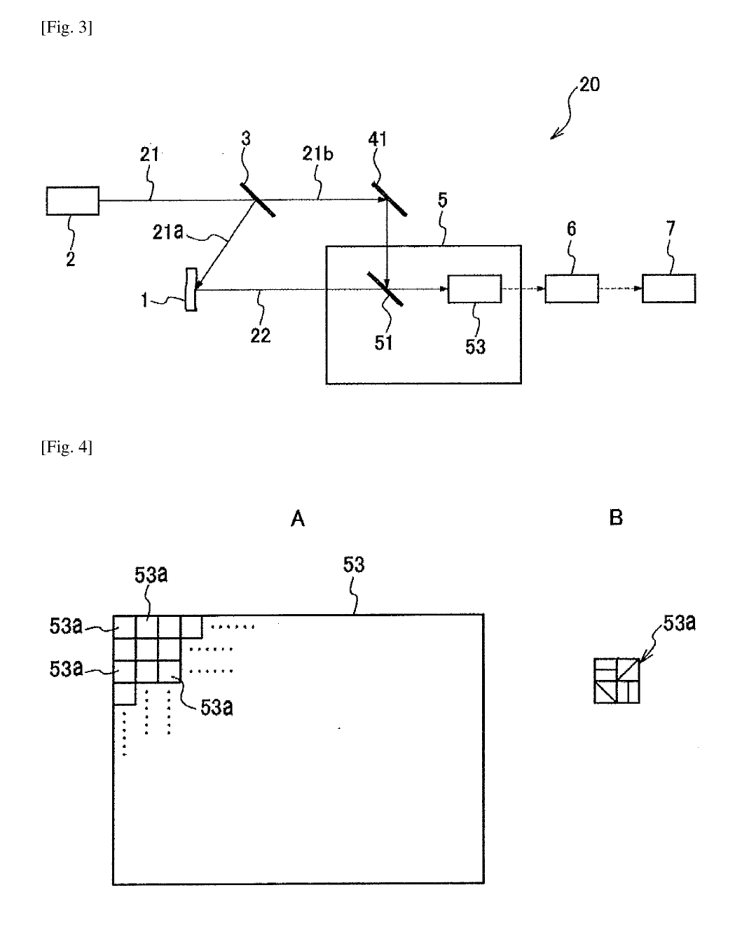

Next, the flow channel imaging apparatus according to the second embodiment of the present disclosure is described. FIG. 3 is a diagram schematically illustrating the configuration of the flow channel imaging apparatus of the present embodiment, and FIG. 4 is a schematic diagram illustrating a configuration example of a polarization imaging element thereof. Here, the same reference numerals are fixed to the same components as those of the flow channel imaging element of the first embodiment described above in FIG. 3, and detailed explanation thereof is omitted.

As illustrated in FIG. 3, a flow channel imaging apparatus 20 of the present embodiment disposes a minor 41 instead of a phase adjustment unit, includes a polarization imaging element 53 in the imaging unit 5 and simultaneously images a plurality of interference fringes with different phase differences between the object light 21a and the reference light 21b. Here, the polarization imaging element 53 is a solid-state imaging element in which one pixel 53a is divided into a plurality of regions for every detection element as illustrated in FIG. 4, and, for example, it is possible to use a polarization camera in which one pixel is divided into four while changing the polarization angles so as to be different from each other by 45°.

Since a polarization imaging element is used in the flow channel imaging apparatus of the present embodiment, it is possible to perform imaging without performing phase shift in an optical path of reference light or object light. Here, other configurations and effects than the above in the flow channel imaging apparatus of the present embodiment are similar to the above-described first embodiment.

Additionally, the present technology may also be configured as below.

(1)

A flow channel imaging apparatus including:

an optical source configured to emit coherent light;

a light separation unit configured to separate the coherent light into object light with which a fluid that flows through a flow channel is irradiated and reference light;

an imaging unit configured to cause scattered light emitted from a region irradiated with the object light and the reference light to interfere with each other, and image an interference fringe;

an image formation unit configured to form a three-dimensional image including a speckle component, with a plurality of images with different phase differences between the object light and the reference light; and

an image processing unit configured to perform addition processing or averaging processing on a plurality of three-dimensional images formed in the image formation unit.

(2)
The flow channel imaging apparatus according to (1), further including: a phase adjustment unit configured to shift a phase of the reference light.
(3)
The flow channel imaging apparatus according to (2), wherein the phase adjustment unit adjusts the phase of the reference light within a range of 0 to 2 pi.
(4)
The flow channel imaging apparatus according to (1), wherein the imaging unit includes a solid-state imaging element in which one pixel is divided into a plurality of regions for every detection element, and simultaneously images a plurality of interference fringes with different phase differences between the object light and the reference light.
(5)
The flow channel imaging apparatus according to any one of (1) to (4), wherein the image formation unit forms a three-dimensional image with four images in which phase differences between the object light and the reference light are different from each other by pi/2.
(6)
The flow channel imaging apparatus according to any one of (1) to (5), wherein the image formation unit forms a three-dimensional image with a plurality of images continuously imaged in a time shorter than a speckle correlation time.
(7)
The flow channel imaging apparatus according to any one of (1) to (6), wherein the flow channel is a blood vessel and the fluid is blood.
(8)
A flow channel imaging method including:
a light separation process of separating coherent light emitted from an optical source into object light with which a fluid that flows through a flow channel is irradiated and reference light;
an image acquisition process of causing scattered light emitted from a region irradiated with the object light and the reference light to interfere with each other, and imaging an interference fringe;
an image formation process of forming a three-dimensional image including a speckle component, with a plurality of images with different phase differences between the object light and the reference light; and
an image processing process of performing addition processing or averaging processing on a plurality of three-dimensional images formed in the image formation process.
(9)
The flow channel imaging method according to (8), further including:
a phase adjustment process of shifting a phase of the reference light before the image acquisition process.
(10)
The flow channel imaging method according to (9), wherein the phase adjustment process adjusts the phase of the reference light within a range of 0 to 2 pi.
( 11)
The flow channel imaging method according to (8), wherein the image acquisition process uses a solid-state imaging element in which one pixel is divided into a plurality of regions for every detection element, and simultaneously images a plurality of interference fringes with different phase differences between the object light and the reference light.
(12)
The flow channel imaging method according to any one of (8) to (11), wherein the image formation process forms a three-dimensional image with four images in which phase differences between the object light and the reference light are different from each other by pi/2.
(13)
The flow channel imaging method according to any one of (8) to (12), wherein the image formation process forms a three-dimensional image with a plurality of images continuously imaged in a time shorter than a speckle correlation time.
(14)
The flow channel imaging method according to any one of (8) to (13), wherein the flow channel is a blood vessel and the fluid is blood.
Alternatively or additionally, the present technology may be configured as below.
(1)
An imaging apparatus comprising:
a light separation unit configured to separate coherent light into object light and reference light, wherein the object light irradiates an object;
an optical element configured to cause interference between:
scattered light emitted from a region irradiated by the object light and the reference light;
an imaging unit configured to image a plurality of interference fringes having different phase differences between the object light and the reference light; and
an image formation unit configured to form, from a plurality of images of the plurality of interference fringes, a three dimensional image including a speckle component comprising a random interference or diffraction pattern.
(2)
The imaging apparatus according to claim 1, further comprising: an image processing unit configured to perform addition processing or averaging processing on a plurality of three-dimensional images formed in the image formation unit.
(3)
The imaging apparatus according to claim 1, further comprising: a phase adjustment unit configured to shift a phase of the reference light.
(4)
The imaging apparatus according to claim 3, wherein the phase adjustment unit is configured to shift the phase of the reference light within a range of 0 to 2 pi.
(5)
The imaging apparatus according to claim 3, wherein the phase adjustment unit comprises a piezoelectric element or an electro-optical element.
(6)
The imaging apparatus according to claim 1, wherein:
the imaging unit includes a polarization imaging element in which one pixel is divided into a plurality of regions for every detection element, and
the imaging unit is configured to simultaneously image the plurality of interference fringes having the different phase differences between the object light and the reference light.
(7)
The imaging apparatus according to claim 6, wherein:
the polarization imaging element comprises a solid-state imaging element, and the imaging unit is configured to simultaneously image the plurality of interference fringes without performing a phase shift in an optical path of the reference light or of the object light.
(8)
The imaging apparatus according to claim 1, wherein the image formation unit is configured to form a three-dimensional image from four images in which phase differences between the object light and the reference light are different from each other by pi/2.
(9)
The imaging apparatus according to claim 1, wherein the image formation unit is configured to form a three-dimensional image from a plurality of images continuously imaged in a time shorter than a speckle correlation time.
(10)
The imaging apparatus according to claim 1, wherein:
the object comprises a fluid that flows through a flow channel.
(11)
The imaging apparatus according to claim 10, wherein the flow channel comprises a blood vessel and the fluid comprises blood.
(12)
The imaging apparatus according to claim 1, further comprising: an optical source configured to emit the coherent light.
(13)
An imaging method comprising:
a light separation process of separating coherent light emitted from an optical source into object light and reference light, wherein the object light irradiates an object;
an image acquisition process of:
causing interference between:
scattered light emitted from a region irradiated with the object light and the reference light, and
imaging a plurality of interference fringes having different phase differences between the object light and the reference light; and
an image formation process of forming, from a plurality of images of the plurality of interference fringes, a three-dimensional image including a speckle component comprising a random interference or diffraction pattern.
(14)
The imaging method according to claim 13, further comprising:
an image processing process of performing addition processing or averaging processing on a plurality of three-dimensional images formed in the image formation process.
(15)
The imaging method according to claim 13, further comprising:
a phase adjustment process of shifting a phase of the reference light before the image acquisition process.
(16)
The imaging method according to claim 15, wherein the phase adjustment process shifts the phase of the reference light within a range of 0 to 2 pi.
( 17)
The imaging method according to claim 13, wherein the image acquisition process: uses a polarization imaging element in which one pixel is divided into a plurality of regions for every detection element, and
simultaneously images the plurality of interference fringes having the different phase differences between the object light and the reference light.
(18)
The imaging method according to claim 17, wherein:
the polarization imaging element comprises a solid-state imaging element, and the image acquisition process simultaneously images the plurality of interference fringes without performing a phase shift in an optical path of the reference light or of the object light.
(19)
The imaging method according to claim 13, wherein the image formation process forms a three-dimensional image from four images in which phase differences between the object light and the reference light are different from each other by pi/2.
(20)
The imaging method according to claim 13, wherein the image formation process forms a three-dimensional image from a plurality of images continuously imaged in a time shorter than a speckle correlation time.
(21)
The imaging method according to claim 13, wherein:
the object comprises a fluid that flows through a flow channel.
(22)
The imaging method according to claim 21, wherein the flow channel comprises a blood vessel and the fluid comprises blood.
(23)
A method comprising:
receiving a plurality of three-dimensional images, wherein each three dimensional image of the plurality of three-dimensional images includes a speckle component comprising a random interference or diffraction pattern;
determining a spatial distribution of speckle intensity in the plurality of three-dimensional images; and
identifying, based on the spatial distribution of speckle intensity, a part of the plurality of three-dimensional images as an object irradiated by coherent light.
(24)
A method comprising:
receiving a plurality of images having different phase differences between an object light of a coherent light and a reference light of the coherent light;
determining a spatial distribution data of speckle intensity based on the plurality of images at least by performing addition processing or averaging processing on the plurality of images; and
identifying, based on the spatial distribution data of speckle intensity, an object irradiated by the coherent light.
(25)
An image processing apparatus comprising:
an image processing unit configured to:
receive a plurality of three-dimensional images, wherein each three dimensional image of the plurality of three-dimensional images includes a speckle component comprising a random interference or diffraction pattern;
determine a spatial distribution of speckle intensity in the plurality of three-dimensional images; and
identify, based on the spatial distribution of speckle intensity, a part of the plurality of three-dimensional images as an object irradiated by coherent light.
(26)
An image processing apparatus comprising:
an image processing unit configured to:
receive a plurality of images having different phase differences between an object light of a coherent light and a reference light of the coherent light;
determine a spatial distribution data of speckle intensity based on the plurality of images at least by performing addition processing or averaging processing on the plurality of images; and
identify, based on the spatial distribution data of speckle intensity, an object irradiated by the coherent light.

Here, effects described in the present specification are merely exemplification and are not limited, and other effects may be provided.

### EXAMPLE

In the following, the effects of the present disclosure are specifically described according to examples of the present disclosure.

### First Example

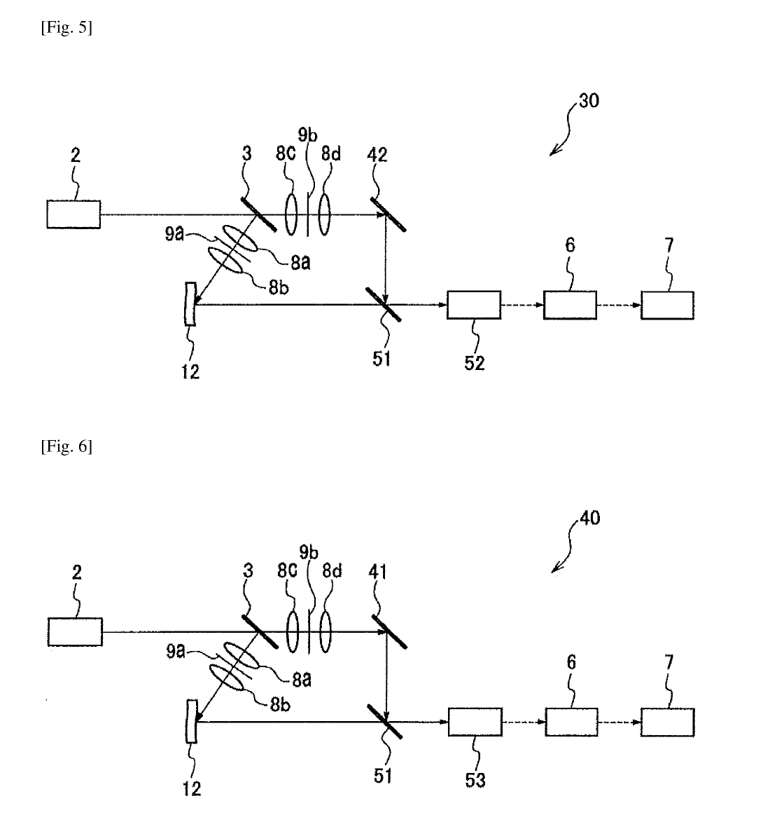

In the first example, a coronary artery of a pig heart was imaged by using the flow channel imaging apparatus of the first embodiment while causing the pig whole blood to flow by a syringe pump at flow velocity of about 1 mm per minute. FIG. 5 is a schematic diagram illustrating the configuration of the flow channel imaging apparatus used in the present example. In the present example, exterior resonance semiconductor laser TEC-520-780-100 (with a wavelength of 780 nm, a single frequency and an output of 100 mW) manufactured by Sacher Lasertechnik was used as the optical source 2. After the coherent light 21 emitted from the optical source 2 was separated into the object light 21a and the reference light 21b by a beam splitter 31, they were subjected to spatial filtering by each lenses 8a to 8d and pinholes 9a and 9b.

The object light 21a was reflected in a half mirror and illuminated a sample 12. A phase shift optical path as illustrated in FIG. 5 was set up in an optical path of the reference light 21b. This optical path was set such that the phase can be changed from 0 to 2 pi by the mirror 41 and a piezoelectric element or an electro-optical element. Object light (scattered light 22) scattered by the sample 12 and the reference light 21b were synthesized by the beam combiner 51. Further, four screens were continuously imaged by a CCD 54 at 100 micro-second intervals in exposure time of 1 micro second while changing the phase from 0, pi/2, pi to 3pi/2, and one three-dimensional image was formed. Such imaging was continuously performed 30 times at 1 m-second intervals, and averaging processing was performed on acquired three-dimensional images.

As a result, a three-dimensional image in which the speckle contrast of the coronary artery part is decreased to about ⅙ as compared with others was acquired. By this means, according to an embodiment of the present disclosure, it has been confirmed that the position of a blood vessel can be specified in a three-dimensional space.

### Second Example

In the second example, a coronary artery of a pig heart was imaged using the flow channel imaging apparatus of the second embodiment described above while causing the pig whole blood to flow by a syringe pump at flow velocity of about 1 mm per minute. FIG. 6 is a schematic diagram illustrating the configuration of the flow channel imaging apparatus used in the present example. As illustrated in FIG. 6, in the present example, the phase shift optical path of the reference light optical path of Example 1 was removed and the CCD was changed to a polarization camera 55. A three-dimensional image was formed by processing outputs with polarization angles different from each other by 45°, which are output from the polarization camera 55. Such imaging was continuously performed 30 times at 1 m-second intervals, and averaging processing was performed on acquired three-dimensional images.

As a result, a three-dimensional image in which the speckle contrast of the coronary artery part is decreased to about ⅙ as compared with others was acquired. By this means, according to an embodiment of the present disclosure, it has been confirmed that the position of a blood vessel can be specified in a three-dimensional space.

It should be understood by those skilled in the art that various modifications, combinations, sub-combinations and alterations may occur depending on design requirements and other factors insofar as they are within the scope of the appended claims or the equivalents thereof.

### REFERENCE SIGNS LIST

1 flow channel

2 optical source

3 light separation unit

4 phase adjustment unit

5 imaging unit

6 image formation unit

7 image processing unit

8a to 8d lens

9a, 9b pinhole

10, 20, 30, 40 flow channel imaging apparatus

11 fluid

12 sample

21 coherent light

21a object light

21b reference light

22 scattered light

31 beam splitter

41 mirror

42 electro-optical element

51 beam combiner

52 imaging element

53 polarization imaging element

54 CCD

55 polarization camera

1. An imaging apparatus comprising:
a light separation unit configured to separate coherent light into object light and reference light, wherein the object light irradiates an object;
an optical element configured to cause interference between:
scattered light emitted from a region irradiated by the object light and the reference light;
an imaging unit configured to image a plurality of interference fringes having different phase differences between the object light and the reference light; and
an image formation unit configured to form, from a plurality of images of the plurality of interference fringes, a three-dimensional image including a speckle component comprising a random interference or diffraction pattern.

2. The imaging apparatus according to claim 1, further comprising:
an image processing unit configured to perform addition processing or averaging processing on a plurality of three-dimensional images formed in the image formation unit.

3. The imaging apparatus according to claim 1, further comprising:
a phase adjustment unit configured to shift a phase of the reference light.

4. The imaging apparatus according to claim 3, wherein the phase adjustment unit is configured to shift the phase of the reference light within a range of 0 to 2 pi.

5. The imaging apparatus according to claim 3, wherein the phase adjustment unit comprises a piezoelectric element or an electro-optical element.

6. The imaging apparatus according to claim 1, wherein:
the imaging unit includes a polarization imaging element in which one pixel is divided into a plurality of regions for every detection element, and
the imaging unit is configured to simultaneously image the plurality of interference fringes having the different phase differences between the object light and the reference light.

7. The imaging apparatus according to claim 6, wherein:
the polarization imaging element comprises a solid-state imaging element, and
the imaging unit is configured to simultaneously image the plurality of interference fringes without performing a phase shift in an optical path of the reference light or of the object light.

8. The imaging apparatus according to claim 1, wherein the image formation unit is configured to form a three-dimensional image from four images in which phase differences between the object light and the reference light are different from each other by pi/2.

9. The imaging apparatus according to claim 1, wherein the image formation unit is configured to form a three-dimensional image from a plurality of images continuously imaged in a time shorter than a speckle correlation time.

10. The imaging apparatus according to claim 1, wherein:
the object comprises a fluid that flows through a flow channel.

11. The imaging apparatus according to claim 10, wherein the flow channel comprises a blood vessel and the fluid comprises blood.

12. The imaging apparatus according to claim 1, further comprising:
an optical source configured to emit the coherent light.

13. An imaging method comprising:
a light separation process of separating coherent light emitted from an optical source into object light and reference light, wherein the object light irradiates an object;
an image acquisition process of:
causing interference between:
scattered light emitted from a region irradiated with the object light and the reference light, and
imaging a plurality of interference fringes having different phase differences between the object light and the reference light; and
an image formation process of forming, from a plurality of images of the plurality of interference fringes, a three-dimensional image including a speckle component comprising a random interference or diffraction pattern.

14. The imaging method according to claim 13, further comprising:
an image processing process of performing addition processing or averaging processing on a plurality of three-dimensional images formed in the image formation process.

15. The imaging method according to claim 13, further comprising:
a phase adjustment process of shifting a phase of the reference light before the image acquisition process.

16. The imaging method according to claim 15, wherein the phase adjustment process shifts the phase of the reference light within a range of 0 to 2 pi.

17. The imaging method according to claim 13, wherein the image acquisition process:
uses a polarization imaging element in which one pixel is divided into a plurality of regions for every detection element, and
simultaneously images the plurality of interference fringes having the different phase differences between the object light and the reference light.

18. The imaging method according to claim 17, wherein:
the polarization imaging element comprises a solid-state imaging element, and
the image acquisition process simultaneously images the plurality of interference fringes without performing a phase shift in an optical path of the reference light or of the object light.

19. The imaging method according to claim 13, wherein the image formation process forms a three-dimensional image from four images in which phase differences between the object light and the reference light are different from each other by pi/2.

20. The imaging method according to claim 13, wherein the image formation process forms a three-dimensional image from a plurality of images continuously imaged in a time shorter than a speckle correlation time.

21. The imaging method according to claim 13, wherein:
the object comprises a fluid that flows through a flow channel.

22. The imaging method according to claim 21, wherein the flow channel comprises a blood vessel and the fluid comprises blood.

23. A method comprising:
receiving a plurality of three-dimensional images, wherein each three-dimensional image of the plurality of three-dimensional images includes a speckle component comprising a random interference or diffraction pattern;
determining a spatial distribution of speckle intensity in the plurality of three-dimensional images; and
identifying, based on the spatial distribution of speckle intensity, a part of the plurality of three-dimensional images as an object irradiated by coherent light.

24. A method comprising:
receiving a plurality of images having different phase differences between an object light of a coherent light and a reference light of the coherent light;
determining a spatial distribution data of speckle intensity based on the plurality of images at least by performing addition processing or averaging processing on the plurality of images; and
identifying, based on the spatial distribution data of speckle intensity, an object irradiated by the coherent light.

25. An image processing apparatus comprising:
an image processing unit configured to:
receive a plurality of three-dimensional images, wherein each three-dimensional image of the plurality of three-dimensional images includes a speckle component comprising a random interference or diffraction pattern;
determine a spatial distribution of speckle intensity in the plurality of three-dimensional images; and
identify, based on the spatial distribution of speckle intensity, a part of the plurality of three-dimensional images as an object irradiated by coherent light.

26. An image processing apparatus comprising:
an image processing unit configured to:
receive a plurality of images having different phase differences between an object light of a coherent light and a reference light of the coherent light;
determine a spatial distribution data of speckle intensity based on the plurality of images at least by performing addition processing or averaging processing on the plurality of images; and
identify, based on the spatial distribution data of speckle intensity, an object irradiated by the coherent light.
